(19) United States
(12) Patent Application Publication
(10) Pub. No.: US 2025/0372223 A1
(43) Pub. Date: Dec. 4, 2025

(54) System and Method for Scanning Patient's Medical Documents Using Artificial Intelligence

(71) Applicant: Seana Noel Strimback, Goodyear, AZ (US)

(72) Inventor: Seana Noel Strimback, Goodyear, AZ (US)

(21) Appl. No.: 19/227,219

(22) Filed: Jun. 3, 2025

Related U.S. Application Data
(60) Provisional application No. 63/655,216, filed on Jun. 3, 2024.

(51) Int. Cl. G16H 15/00; G16H 10/40; G16H 10/60; G16H 40/00; G16H 80/00

(52) U.S. Cl. CPC G16H 15/00; G16H 10/40; G16H 10/60; G16H 40/00; G16H 80/00

(57) ABSTRACT

The invention provides a system and method for consolidated patient data reporting in healthcare facilities utilizing artificial intelligence for decline and terminal status assessment. The system includes a data input module configured to receive patient data from multiple healthcare providers across different facilities in various formats, and a processing module for processing and amalgamating stored patient data. An artificial intelligence module utilizes optical character recognition, natural language processing, and specialized algorithms to scan unstructured medical documents and extract pertinent patient data focused on deterioration markers including weight trends, laboratory results, symptom patterns, functional changes, and healthcare utilization patterns. A reporting module generates consolidated patient data reports providing real-time updates and summaries highlighting significant decline trends and terminal indicators. The system includes modal-based human-in-the-loop validation, comprehensive HIPAA compliance measures, and tiered access control for enhanced security and data integrity.

### CROSS REFERENCE TO RELATED APPLICATIONS

This application claims benefit from currently pending U.S. Provisional Application No. 63/655,216 titled “A System and Method for Scanning Patients Medical Documents Using Artificial Intelligence” and having a filing date of Jun. 3, 2024, all of which is incorporated by reference herein.

### FIELD OF THE INVENTION

This technology pertains to the field of healthcare informatics, specifically focusing on systems for consolidating and reporting patient data to healthcare workers and even more specifically a system that can collect patient data from multiple sources, pull the pertinent information from the data and compile it in a readable report for the healthcare worker.

### BACKGROUND OF THE INVENTION

In the current healthcare landscape, managing and interpreting patient data is a complex and challenging task. Healthcare providers across multiple facilities or a single facility generate a vast amount of patient data, including demographics, allergies, weight and height history, infection occurrences, laboratory results, medical imaging results, vital signs, respiratory status, activities of daily living, symptom tracking, diagnosed diseases, hospitalizations, emergency service admissions, communications, historical documents, and general health trends. This data is crucial for patient care, but its sheer volume and diversity can make it difficult to manage and interpret effectively.

Traditionally, patient data has been stored and managed in a fragmented manner across different healthcare facilities and through different electronic health record platforms. The data fragmentation is further compounded by the varied formats used to store patient information, ranging from PDFs, text, voice calls and structured databases to physical documents. The disjointed nature of data acquisition poses substantial challenges in healthcare settings that necessitate prompt and accurate decision-making. Healthcare providers frequently need to contact multiple sources to compile a patient's comprehensive medical history, often relying on communication methods such as phone calls, faxes, and emails. Each method involves delays and risks of data miscommunication or loss, adversely affecting the timeliness and reliability of the assembled patient data. These delays can lead to decision-making based on incomplete or outdated information, potentially resulting in adverse patient outcomes, redundant testing, and inefficient resource allocation.

For example, a healthcare provider in a hospital or hospice situation may not have access to a patient's complete medical history, leading to potential misdiagnoses or inappropriate treatments. Furthermore, the lack of a unified data repository can make it difficult to identify significant trends and vital information in patient care. The process of inputting patient data, scanning through emails, looking through patient charts can be prone to errors. Healthcare workers often have to manually input data into various systems, which can be time-consuming and are prone to errors. Errors in patient data can have serious consequences, including incorrect diagnoses, inappropriate treatments, and even patient harm. Moreover, patient data is sensitive and needs to be protected to maintain patient privacy and comply with regulations.

Traditional systems may not have robust security and access control mechanisms, potentially exposing patient data to unauthorized access. Healthcare providers need to be able to generate consolidated patient data reports that provide real-time updates and summaries of patient data that can help the healthcare provider make an informed decision on the patient's treatment plan or if they are ready for hospice. These reports are crucial for monitoring patient health, making treatment decisions, and identifying health trends and generating these reports can be challenging due to the fragmented nature of patient data and the lack of effective data processing tools.

The operational inefficiencies arising from the scattered nature of patient data directly impact patient care quality. The inability to promptly retrieve and interpret comprehensive medical histories can lead to critical oversights, such as unmonitored chronic conditions, missed trends indicative of potential health issues, and incomplete information for clinical decision-making. Additionally, inconsistencies in the interpretation of medical terminology and records among different healthcare providers introduce another complexity layer, further hindering integrated and responsive patient care delivery. Further medical documents are very large, often 50-100 pages, and include many pieces that are unnecessary and repeating information that can be difficult to navigate. The current documents provided are in various formats and the verbiage is not patient-friendly and can only be adequately interpreted by a medical worker in that specific field or from that specific medical facility. Although medical documents have been digitized, access to them and the ability to obtain and interpret them remains out of reach to most of the population.

Given these challenges, there has been a growing trend towards developing centralized real-time reporting systems to unify patient data from multiple sources. Such systems aim to provide healthcare workers with standardized, updated, and comprehensive views of patients' health statuses, irrespective of their locations or specific facility cultures. Technologies like Software as a Service (SaaS)-based applications have shown promise by offering remote access through web-based interfaces, secure data transmission via standard internet protocols, and robust patient privacy through end-to-end encryption. Despite these advancements, achieving a fully integrated and secure healthcare data management system that dynamically adapts to patient needs and evolving healthcare practices remains a significant endeavor.

Alongside data integration, maintaining the security of sensitive healthcare information is crucial. Emerging systems must incorporate robust security measures, including secure socket layering (SSL) and end-to-end encryption, to safeguard data privacy. Role-based access control mechanisms are essential for maintaining data integrity and confidentiality, tailored to various user roles such as administrators, healthcare workers, and patients. The architectural flexibility to operate independently of existing IT setups while offering future system integration capacity is pivotal for these systems' scalability and successful implementation across diverse healthcare environments.

Scalability and adaptability to different healthcare settings are vital considerations. A practical system must cater to diverse environments by addressing specific requirements of varied healthcare providers. This includes providing mobile data entry solutions compatible with devices like smartphones and tablets, ensuring data entry is convenient and accurate for healthcare workers. Systems should generate actionable insights from patient data, presenting concise reports amalgamating critical information from extensive medical histories, thus increasing efficiency, reducing administrative burdens, and facilitating rapid medical decision-making.

While significant strides have been made in digitizing patient records and managing healthcare data, the potential for fully integrated, real-time patient data aggregation and reporting systems is yet to be fully realized. The inherent complexities of existing disparate systems highlight the necessity for comprehensive solutions that enhance patient care through streamlined data access and interpretation. A robust system that minimizes the time and effort required for managing patient information will empower healthcare workers to deliver timely and effective patient care, resulting in improved patient outcomes and operational efficiencies within healthcare settings.

Therefore, there is a need for a system that addresses these shortcomings by providing seamless integration of patient data from multiple sources, ensuring real-time access to comprehensive, up-to-date patient information for healthcare and hospice workers. Such a solution should incorporate advanced algorithmic and AI components for data interpretation, robust security measures, and scalable architecture adaptable to diverse healthcare settings, ultimately enhancing patient care quality and operational efficiency in the healthcare.

### BRIEF SUMMARY OF THE INVENTION

The present invention provides a system for consolidating patient data reporting across multiple healthcare facilities. The system collects patient information from various healthcare providers and different medical facilities, storing all this data in a single, unified repository. An advanced processing module works together with an artificial intelligence component to analyze and combine the stored patient data, with the artificial intelligence specifically designed to identify significant trends over time within the patient information. The system then generates consolidated patient reports that provide healthcare workers with real-time updates and comprehensive summaries of patient data. Data in the consolidated report may include a source tag that identifies exactly where that information originated, allowing healthcare workers to quickly locate and review the original source document when they need to investigate any unusual findings or potential errors detected in the consolidated report.

The invention also encompasses a method for creating these consolidated patient reports that focuses on trend identification and analysis. The process begins by collecting patient data from multiple healthcare providers across different facilities and storing this information in a unified database. The system uses sophisticated artificial intelligence algorithms to process and organize the stored data, specifically designed to identify significant trends over time within the patient information. When the system encounters conflicting or duplicate information, it tags these data points with links that direct healthcare workers to the exact source of the conflicting data, enabling clinical review and resolution. The patient data is organized into specific categories including demographics, allergies, weight and height history, infection occurrences, laboratory results, medical imaging results, vital signs, respiratory status, activities of daily living, symptom tracking, diagnosed diseases, hospitalizations, emergency service admissions, communications, historical documents, and general health trends. The artificial intelligence identifies significant trends within these individual categories and then analyzes relationships between trends across different categories to create more comprehensive insights. The system generates detailed reports that provide real-time updates and summaries of patient data, and allows authorized clinicians to review and edit the information after their analysis, with these edits being reflected in the updated consolidated reports.

Additionally, the invention includes a specialized method for handling both structured and unstructured patient data with enhanced trend analysis capabilities. The system can identify different types of data formats within patient records and uses an artificial intelligence document parser to extract raw information from unstructured sources such as narrative notes, scanned documents, and various file formats. This extracted information is then analyzed and filtered to convert it into structured data that can be systematically stored in the unified repository. The system processes this organized data using artificial intelligence algorithms specifically designed to search the repository for significant trends over time in the patient data. Like the general method, the patient information is categorized into specific groups, and the system identifies significant trends within individual categories as well as relationships between trends across different categories. The system generates consolidated reports that provide real-time updates and summaries of patient data, incorporates tiered access control for security, allows clinicians to edit reviewed data with those changes reflected in the consolidated reports, and employs conflict resolution workflows to maintain data integrity throughout the entire process.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims. Aspects and applications of the invention presented here are described below in the drawings and detailed description of the invention.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for, and will also recite the word “function” (i.e., will state “means for performing the function of . . . ”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112(f). Moreover, even if the provisions of 35 U.S.C. § 112(f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF THE DRAWINGS

A more complete understanding of the present invention may be derived by referring to the detailed description when considered in connection with the following illustrative figures. In the figures, like reference numbers refer to like elements or acts throughout the figures.

FIG. 1 shows record creating screen of the system in accordance to one or more embodiments;

FIG. 2 shows patient's history of the system in accordance to one or more embodiments;

FIG. 3 shows symptoms, diagnoses, and hospitalizations of a patient of the system in accordance to one or more embodiments;

FIG. 4 shows example record lab clinical measurements of the system in accordance to one or more embodiments;

FIG. 5 shows vitals, infection occurrences, notes and ADL's of the patient of the system in accordance to one or more embodiments;

FIG. 6 shows legend screen of the system in accordance to one or more embodiments;

FIG. 7 shows sample patient screen of the system in accordance to one or more embodiments;

FIG. 8 shows sample labs screen of a patient of the system in accordance to one or more embodiments;

FIG. 9 shows labs clinical measurement of a patient of the system in accordance to one or more embodiments; and

FIG. 10 shows example record creation screen where a HCW can enter data for a patient of the system in accordance to one or more embodiments.

Elements and acts in the figures are illustrated for simplicity and have not necessarily been rendered according to any particular sequence or embodiment.

### DETAILED DESCRIPTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

The present invention relates to a system for consolidated patient data reporting in healthcare facilities. The system comprises a data input module, a unified data repository, a processing module, an artificial intelligence module, and a reporting module. In a particular embodiment, the system specifically utilizes Claude transformer architecture for testing procedures, incorporating custom inputs for data synthesis and retrieval with a developed training data structure that includes medical documents along with FHIR (Fast Healthcare Interoperability Resources) mapping.

The data input module can configure to receive patient data from multiple healthcare providers across different facilities. This module is designed to facilitate the collection of a wide range of patient data, including but not limited to demographics, allergies, weight and height history, infection occurrences, laboratory results, medical imaging results, vital signs, respiratory status, activities of daily living, symptom tracking, diagnosed diseases, hospitalizations, emergency service admissions, communications, historical documents, and general health trends.

Medical records in modern healthcare systems contain two different types of data that present unique challenges for data processing and analysis. Structured data represents information that is in digital systems using predefined fields with specific format conventions and constraints. This type of data is highly organized and follows standardized formats that make it easily searchable, sortable, and analyzable by computer systems. Examples of structured data in medical records include patient demographics entered into specific fields such as name, date of birth, address, and phone number, vital signs recorded in designated numeric fields like blood pressure readings of 120/80, temperature of 98.6° F., and heart rate of 72 beats per minute, laboratory results with standardized units and reference ranges such as glucose levels of 95 mg/dL and hemoglobin of 14.2 g/dL, medication lists with standardized drug names, dosages, and frequencies like Metformin 500 mg twice daily, ICD-10 diagnostic codes such as E11.9 for Type 2 diabetes mellitus without complications, CPT procedure codes for billing and documentation, allergies selected from dropdown menus with severity levels, and insurance information in standardized fields. Structured data follows consistent formatting rules and data types, can be easily queried using database search functions, enables automated calculations, trending, and statistical analysis, facilitates interoperability between different healthcare systems, supports standardized reporting and quality metrics, and allows for efficient data validation and error checking.

Unstructured data consists of information that exists in free-form text or document formats without predefined fields or standardized organizational structure. This data often contains rich, detailed clinical information but is much more challenging for computer systems to process and analyze automatically. Examples of unstructured data in medical records include physician progress notes written in narrative format describing patient encounters, nursing assessments and observations documented in free-text fields, discharge summaries that provide comprehensive overviews of hospital stays, consultation reports from specialists containing detailed clinical reasoning, radiology reports describing imaging findings in descriptive language, pathology reports with detailed microscopic descriptions, emergency department notes documenting the sequence of events and clinical decision-making, handwritten notes that have been scanned into the system as PDF documents, voice-to-text transcriptions of dictated clinical notes, historical paper records that have been digitized through scanning, clinical correspondence between providers, and patient-reported symptoms and concerns documented in narrative form.

Unstructured data contains natural language with medical terminology, abbreviations, and clinical shorthand, includes temporal relationships and clinical reasoning that may not be captured in structured fields, often contains critical information about patient symptoms, physical examination findings, and clinical impressions, may include relevant family history, social history, and lifestyle factors, can contain information about treatment responses, medication effectiveness, and adverse reactions, and often includes provider observations about patient behavior, compliance, and functional status.

In practice, most medical records contain both structured and unstructured data, creating a complex information landscape. A single patient encounter might generate structured vital signs and lab values entered into EMR fields, unstructured physician notes describing the clinical assessment and plan, structured medication orders with specific dosing instructions, unstructured nursing notes about patient response and care interventions, structured billing codes for procedures performed, and unstructured radiology reports describing imaging findings.

The coexistence of structured and unstructured data creates significant challenges for comprehensive patient data analysis. Structured data can be easily aggregated across patients for population health analysis, supports automated clinical decision support and alerts, enables efficient quality reporting and regulatory compliance, and facilitates research through large dataset analysis. However, unstructured data contains valuable clinical context often missing from structured fields, requires advanced natural language processing to extract meaningful information, may include critical details about patient decline, symptom progression, or treatment failures, and often contains nuanced clinical observations that don't fit into predefined categories.

The system is equipped to process diverse medical data formats including PDF, Excel, XML, HL7, FHIR, and proprietary formats, with the capability to develop both integrated and custom solutions for Electronic Medical Record (EMR) transactions that are not currently supported. The data input module includes a mobile application and a web-based interface, allowing healthcare workers to input patient data. The mobile application and web-based interface are designed to be user-friendly, with forms and drop-down fields that minimize input errors. This ensures that the data collected is accurate and reliable, which is crucial for the effective operation of the system.

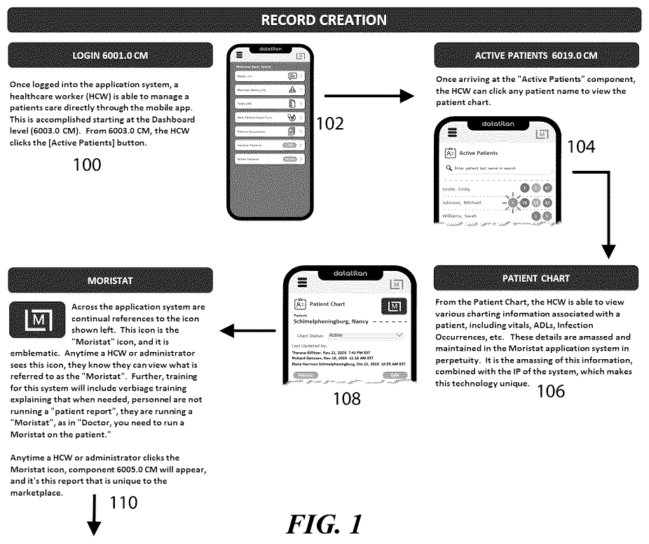

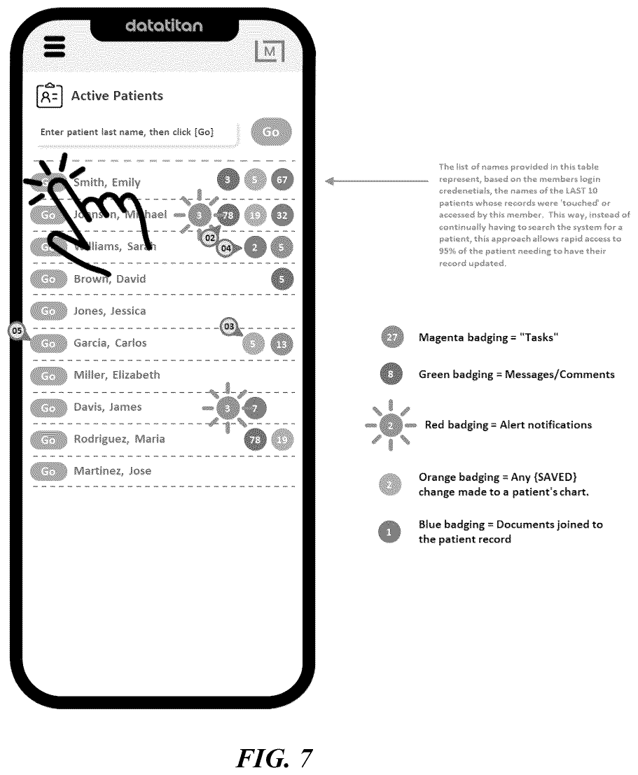

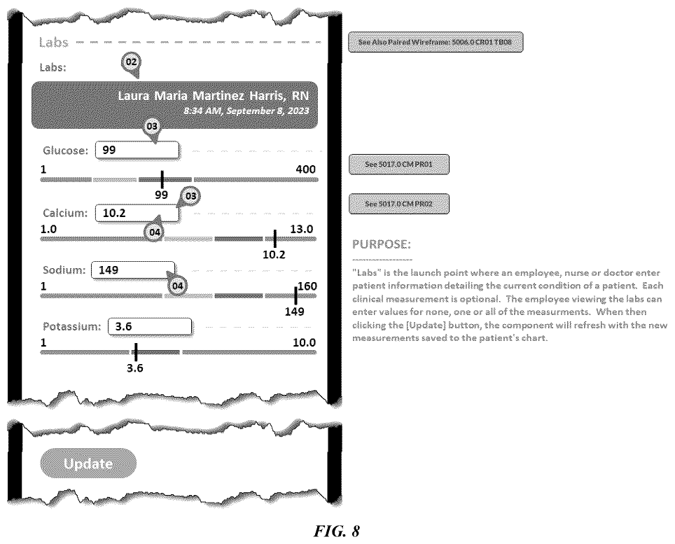

Referring to FIG. 1 at step 100 a Healthcare Worker (“HCW”) or user can log into the application wherein the HCW is able to manage at least one patient care. At the dashboard level of the application the HCW can access a menu that can be such as, but not limited to taking notes, receiving alerts, receiving and setting tasks, inputting new patient information, seeing patient documents, looking at inactive patients and active patients at 102 wherein the menu can be seen and referenced across the whole platform by the HCW. The HCW can click on active patients which leads to an active patient screen at 104. The active patient screen can show all the active patients wherein the HCW can click on any patient name to view the patient chart as shown in FIG. 7. In certain embodiments the active patients can be sorted by such as, for example, alphabetically, newest patient, oldest patient, or the like.

In embodiments, at 106 from the patient chart, the HCW can view various charting information associated with the patient such as, for example, vitals, activities of daily living (“ADL”) including trends and critical changes, infection occurrences, laboratory results, medical imaging results, vital signs and respiratory status, symptom tracking and trends, diagnosed diseases including historical, current and new and changing disease processes, hospitalization including record of how many admissions to emergency services, hospital stays, and the details of care received and how they affect patient status, communications between healthcare providers and staff directly involved in patient care, patient historical documents including uploaded historical data submitted for review and addition to the record and new documents that are received for ongoing review, and documents relating to ongoing/future care and patient wishes such as medical power of attorney, end-of-life planning resources, or decision-making materials patients provide and submit and general health trends and decline relating to physical or medical measurements, and the patient stated needs or changes (collectively referred to as “Charting Information”). All the Charting Information can be compiled and then kept within the application for each patient both active and inactive.

At 108, the patient chart can show the patient's name, chart status such as active or inactive, last updated by and can allow the HCW to see any of the patient Charting Information. Across the application an icon symbol can be shown at 110. The icon symbol allows the HCW or administrator to scan all the uploaded Charting Information for the patient which can be called a search wherein the search uses Artificial Intelligence to scan Charting Information for pertinent information about the patient. The system's analytical approach is centered on identifying signs, symptoms, and trends suggestive of deterioration, as well as markers indicative of terminal status, focusing specifically on data pertinent to these defined areas rather than providing a comprehensive patient health record. The system utilizes PostgreSQL, leveraging its JSON capabilities and optimization techniques, to implement a unified and high-performance data management strategy that enables efficient handling of both structured and semi-structured data.

The unified data repository is where the patient data received from the data input module is stored. This repository is designed to store a large volume of data, ensuring that all patient data collected from different healthcare providers and facilities can be accommodated. The unified data repository is also designed to facilitate easy access and retrieval of data, which is crucial for the processing and analysis of the data. The processing module is responsible for processing and amalgamating the stored patient data. This module uses advanced algorithms to process the data, ensuring that the data is organized and structured in a way that facilitates easy analysis and interpretation. The processing module can also amalgamate the data, combining data from different sources into a unified format. This ensures that the data is consistent and coherent.

The artificial intelligence module can also identify trends over time in patient data that are especially significant in evaluating the status of a patient. Significant trends include:
a) Weight Trend Analysis: Identifies 5% weight loss over three months, or any equivalent ratio of timing and percentage, not related to diet, diuretic therapy, edema, or intentional change, using algorithmic separation to distinguish actual trends from expected factors.
b) Decreasing Objective Measures: Analyzes physical measurements of patient landmarks such as mid-arm, abdomen, and thigh to detect downtrends not expected from medications or lifestyle changes.
c) Laboratory Result Analysis: Processes all laboratory results to identify specific data trends such as decreasing levels of albumin or cholesterol, increasing pCO2, decreasing pO2 in relation to algorithmically defined terminal diagnoses such as COPD or Cardiac Disease, and changes in kidney and liver values linked to corresponding terminal diagnoses.
d) Nutritional Intake Assessment: Analyzes nutritional intake information including caloric value, dietary methods, texture of foods, symptoms associated with decreased oral intake, dysphagia, swallow evaluations, and relevant neurological diagnoses.
e) Symptom Tracking: Processes all text fields including clinical narratives to identify respiratory symptoms, gastrointestinal symptoms, pain patterns, and other indicators frequently not coded or diagnosed.
f) Functional Changes Assessment: Evaluates signs, symptoms, and scoring methods including fatigue, weakness, unresponsiveness to therapy, Palliative Performance Scale scores, and changes in activities of daily living.
g) Healthcare Utilization Analysis: Tracks medical encounters to identify increased usage or recurrent episodes indicating early signs of decline.
h) Integumentary Analysis: Conducts comprehensive analysis of all integumentary-related declines cross-referenced with nutritional data.
i) Diagnostic Review: Identifies terminal diagnoses through both coded records and physician narratives using algorithmically defined terminal conditions and associated comorbidities.

The artificial intelligence module employs a distinct and internally developed assessment framework to identify individuals exhibiting signs of decline or approaching terminal stages of life, constructed upon well-established indicators derived from extensive research and expert consensus.

They system may also identify relationships between different trends that are especially indicative of decline or approaching terminal stages of life. For example, the physical measurements of patient landmarks may be cross-examined with various data points, including medications that may influence the data and changes in lifestyle. Or increasing pCO2, decreasing pO2 in relation to algorithmically defined terminal diagnoses such as COPD or Cardiac Disease and changes in kidney and liver values, particularly when assessed algorithmically, should be linked to a corresponding terminal diagnosis, such as Chronic Renal Failure or Liver Diseases.

If applicable, this information is presented in the report, which indicates any signs of decline. A decline parameter may be misinterpreted as a positive change, such as a decrease in cholesterol levels, even when the overall patient data indicates a negative trend. The system can identify specific changes in relation to other data points. Medical providers may review these details, but they are often not aggregated, assessed, or reported in a way that facilitates timely application to patient assessment or treatment for appropriate intervention and prevention. By cross-referencing various documents and events, such as hospitalizations and treated illnesses, we can identify trends or changes that may have been noted earlier in the process, even if the data appears unrelated.

Using a computer system and AI, the AI can create the critical points and trends by for example, a digitization and an Optical Character Recognition (“OCR”) which can scan the Charting Information creating digital copies. The system's operational extraction accuracy target is 98%, with an operational objective to achieve extraction periods of under five minutes. All data undergoes analysis compatible with multi-format document processing, with each data point tagged with its source for precise location and review of anomalies. The OCR can convert the scanned images of the text into machine readable text wherein the OCR can convert handwritten and printed notes into digital text that can be processed by AI. The Chart Information can be preprocessed wherein the Chart Information can be cleaned to remove any noise such as irrelevant characters or formatting issues. The Chart Information can be normalized by converting the Chart Information into a consistent format, such as, for example, uniform date formats and medical terminology.

The system implements data normalization and standardization across different healthcare systems by adhering to a data definition framework aligned with prevalent terminology and declarative language sanctioned across all medical disciplines. The platform facilitates data standardization by processing inconsistent data formats, including PDFs lacking field recognition and narrative reports, into a uniform structure using FHIR schema implementation. A natural language processing (“NLP”) can tokenize the Chart Information by breaking it down into individual words or phrases for analysis. The system can then identify and classify key entities such as patient names, dates, medications, diagnoses, symptoms, or the like through a Named Entity Recognition (“NER”). A Part-of-Speech Tagging can be used to identify the grammatical parts of speech in the text to understand the context of the words. A syntax and semantic analysis can be used to understand the structure and meaning of sentences and to interpret complex medical information.

The Chart Information can then be extracted by a pattern matching and rules-based systems wherein using predefined patterns and medical knowledge rules to extract specific information such as diagnoses, treatments, and lab results. A machine learning models can be used to train supervised learning models on annotated medical records to recognize and extract relevant information. This can include techniques such as support vector machines (SVM), decision trees, or deep learning methods like convolutional neural networks (CNNs) and recurrent neural networks (RNNs). For conflicting or duplicate data identification and resolution, the system employs model-based validation with duplicate data initially refined and then clearly identified and editable via a universal icon button across all data sources. All data necessitates review by credentialed clinicians for analytical purposes, including determination of conflicting reports and assessment of potential alterations in patient condition. Data assessed as clinically conflicting may be excluded from reports and documented accordingly while maintaining the official record integrity.

A data integration and structuring including data mapping can be used for aligning extracted information with standardized medical ontologies and databases (e.g., ICD-10 for diagnoses, SNOMED CT for clinical terms). The Chart Information can be structured by organizing the extracted information into structured formats such as databases or spreadsheets for easy querying and analysis. An analysis and insights generation can be used for aggregating and summarization the patient data to provide a summary of key information such as medical history, current medications, and recent test results. Once the pertinent Chart Information is analyzed and summarized predictive analytics can be used for applying predictive models to identify potential health risks, suggest treatment options based on historical data, or determine whether the patient is ready for hospice. Anomalies can be detected by identifying unusual patterns or deviations in patient records that may indicate errors or significant medical events.

Once the above is complete the report can be shown through a visualization tool wherein a dashboard with visual representations of the patient data for easy interpretation HCW can be shown through natural language generation (“NLG”) by generating readable summaries and reports from the extracted and analyzed data, making it accessible to non-technical users.

The system architecture utilizes Django REST Framework, leveraging Django for web development and the REST Framework for Web APIs, enabling scalable backend services for data management and secure API endpoints. The mobile and web interfaces integrate with the backend through this separation of concerns architecture, enhancing maintainability and development efficiency.

The reporting module is responsible for generating and presenting a consolidated patient data report. This report provides real-time updates and summaries of the patient data, ensuring that healthcare providers and facilities have access to the most up-to-date and accurate information. The system offers robust integration capabilities through Application Programming Interfaces (APIs), enabling seamless data exchange and interoperability with various healthcare systems and third-party applications, with flexibility to adapt and align with diverse Electronic Medical Record models and configurations.

The reporting module transforms processed data to highlight significant trends and vital information in patient care, enhancing the value and usefulness of the reports. The system can include an error identification module for detecting and flagging discrepancies or errors in the patient data wherein this module can use advanced algorithms and AI to scan the data, identifying and flagging any discrepancies or errors, which can ensure that the data used by the system is accurate and reliable, which is crucial for the generation of accurate and meaningful reports.

The system implements comprehensive HIPAA compliance measures including robust encryption for all PHI both at rest and in transit using industry-standard protocols, comprehensive audit trails for all access and modifications, stringent access controls adhering to the principle of least privilege, and adherence to all mandated administrative, physical, and technical safeguards. The system performs annual third-party HIPAA evaluations and ensures compliance with external user market requirements. The system can further include a security and access control module wherein this module can provide tiered access control, with levels including administrative access, facility administration, and patient access. This ensures that the data stored in the system is secure and that access to the data is controlled and regulated, protecting the privacy and confidentiality of the patient data. In embodiments a legend can be displayed for the HCW allowing them to see the risk assessment level for the patient at 140 and the at step 138 the codes for each numbering.

The system employs a modal-based human-in-the-loop validation system, leveraging specialized data comparison and coupling techniques to facilitate enhanced analysis of correlated events that are either segregated in standard reports or otherwise overlooked. In embodiments there can be three levels of accessibility wherein level one requires that the administrator creates and provides access level control to a healthcare facility. Level two can designate the healthcare facility access to the application and can allow them to enter the HCW that work in their facility, but only after a facility administrator has granted access to them. Usernames are required to be corporate-issued email addresses. The access granted by the facility to the HCW is provided to a specific patient's data. Access to a patient's chart is a secondary level of access and is granted only to the HCWs assigned to that patient. No global patient access is granted to a HCW in their healthcare facility, or to the system as a whole. It must be assigned on a patient-by-patient basis. This user or patient access can be revoked easily and quickly by any administrator of the system or healthcare facility administrator. Level three can access to application system is given to patients directly. However, patients are required to enter a username, password, and a patient identification number issued by the application system. The patient cannot enter information into their chart, change, or view live data. The access granted to a patient can only to view processed historical documents and reports to assist in their own communication with their healthcare providers or personal education on their healthcare-related data.

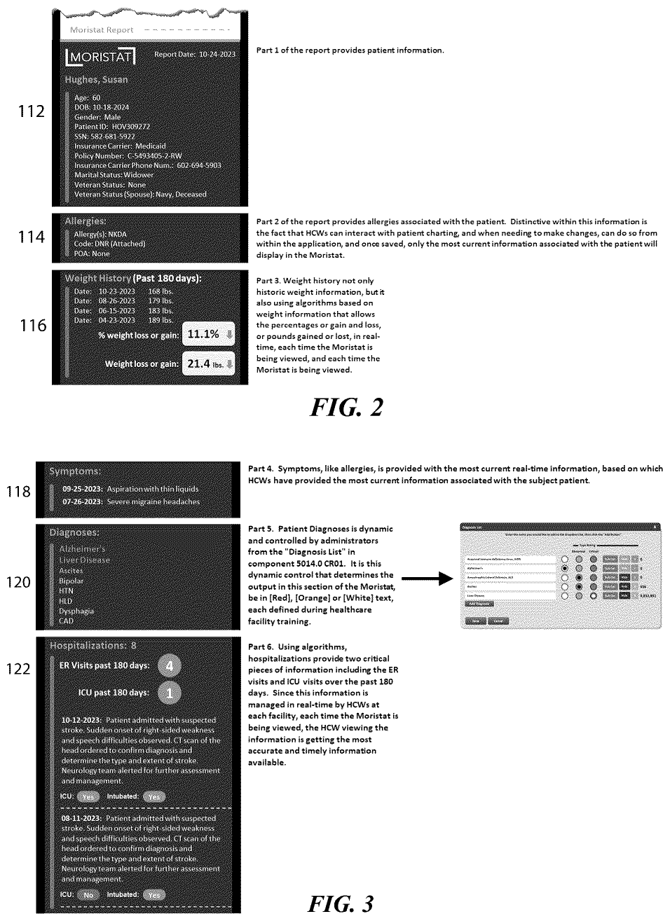

Referring to FIG. 2, at 112 shows an example report with the patient's information such as, for example, age, date of birth, gender, patient ID, social security, insurance carrier, policy number, insurance carrier phone number, marital status, veteran status, or the like. At 114, The patient report can list out what type of allergies the patient has, the codes such as, for example, do not resuscitate (“DNR”), Do Not Intubate (“DNI”), Allow Natural Death (“AND”), Comfort Measures Only (“CMO”), or the like, and whether the patient has a durable power of attorney for health care, living will, or the like. At 116, the patient's historical weight history can be shown with the percentage of weight loss or gain and/or weight loss or gain, which can be shown in real-time each time the report is viewed.

Referring to FIG. 3, at 118 the patient's symptoms, like allergies, can be provided with the most current real-time information, based on which the HCWs have provided the most current information associated with the subject patient such as the date and symptoms. For example, one Sep. 25, 2023 the patient had aspiration with thin liquids. At 120 the patient diagnoses can be dynamic and controlled by an administrator from the “Diagnosis List.” The diagnoses can be such as, for example, Alzheimer, liver disease, ascites, bipolar, HTN, HLD, dysphagia, CAD, or the like. The administrator can have an interface which can give up to three levels of access which can be assigned to all users. These include such as, for example, healthcare administrator access, healthcare worker access, direct patient access, and the like, and through these levels of access each persona type is given permissions and access to different types of operational control. The operational controls can provide users access to data and functions that can provide access to faster and better care.

At 122, from the search the patient's hospitalizations can be displayed along with critical pieces of information that can include emergency room (“ER”) and intensive care unit (“ICU”) visits over the past 180 days which can be managed in real-time by the HCW at each facility, each time the report is being viewed. The HCW viewing the information can get the most accurate and timely information through the above analysis and information and then information can then be displayed to the HCW. For example, the application can display the number of ER visits in the past 180 days and the ICU past 180 days and can display the date and the reason for each ER visit and whether it included a visit to the ICU, and whether the patient was intubated.

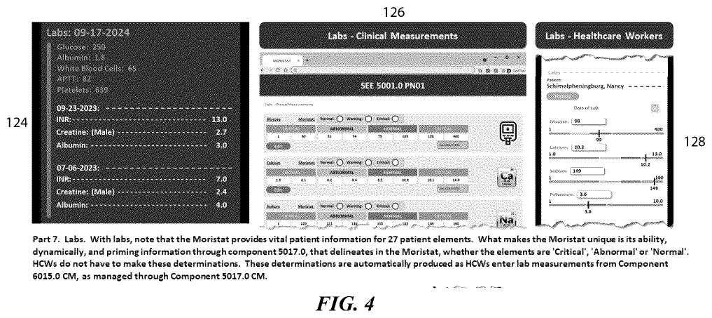

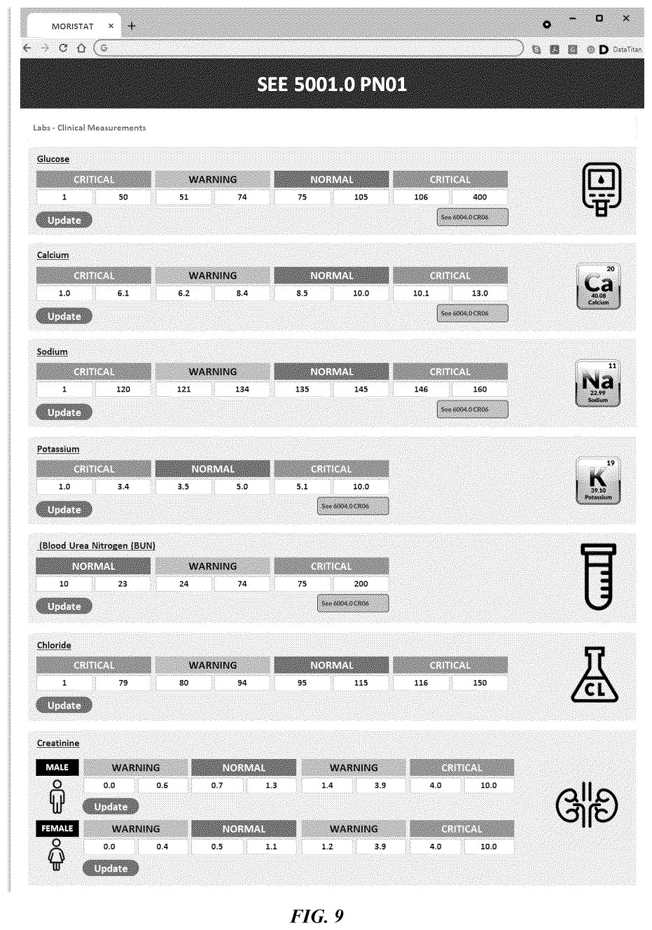

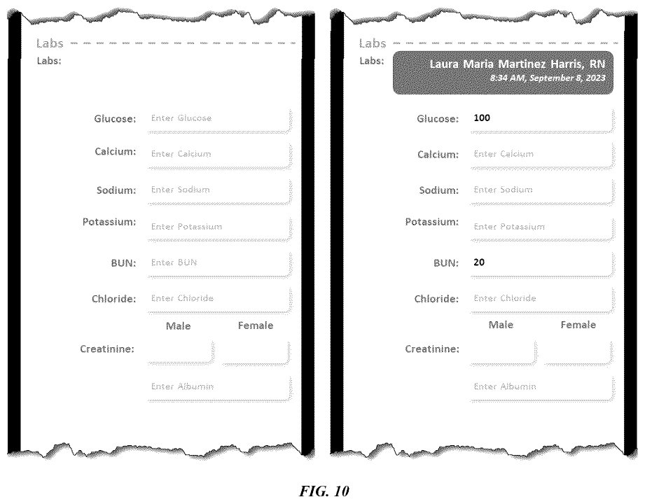

Referring to FIG. 4, at 124 a patient's labs can be displayed wherein the application can search and pull such as, for example, the date the labs were taken, glucose levels, albumin, white blood cells, APTT, platelets, INR, creatine, albumin for both male and female. At 126 the patient's lab clinical measurements can be searched and displayed. The clinical measurements can be such as, for example, glucose, calcium, sodium, potassium, blood urea nitrogen, chloride, creatinine, albumin, hemoglobin, hematocrit, white blood cells, red blood cells, platelets, troponin, prothrombin time, international normalized ratio, PTT (heparin), Activated partial thromboplastin time, pH, Po2, PCO2, HCO3, B-type natriuretic peptide, glomerular filtration rate, CD4, AST, ALT, or the like. The search can compile all the patient's lab results and determine whether the patient elements are critical, abnormal, or normal as shown in FIG. 9. The patient's labs can be automatically produced and then displayed as HCWs enter lab measurements into the application as shown in FIG. 10. At step 128, the labs can allow an employee, nurse or doctor to enter the patient's information detailing the current condition of the patient wherein each clinical measurement is optional. The employee, nurse or doctor viewing the labs can enter values for none, one or all of the measurements wherein when the employee, nurse or doctor can click update button and the component can refresh with the new measurements saved to the patient's chart as shown in FIG. 8.

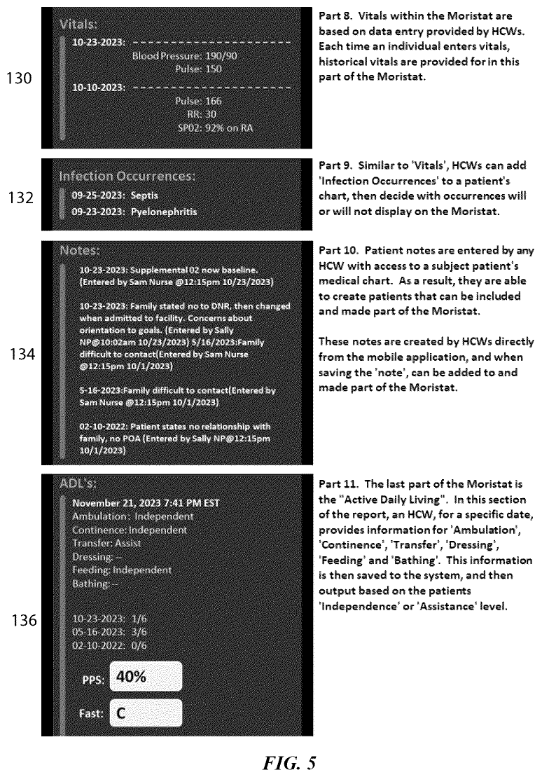

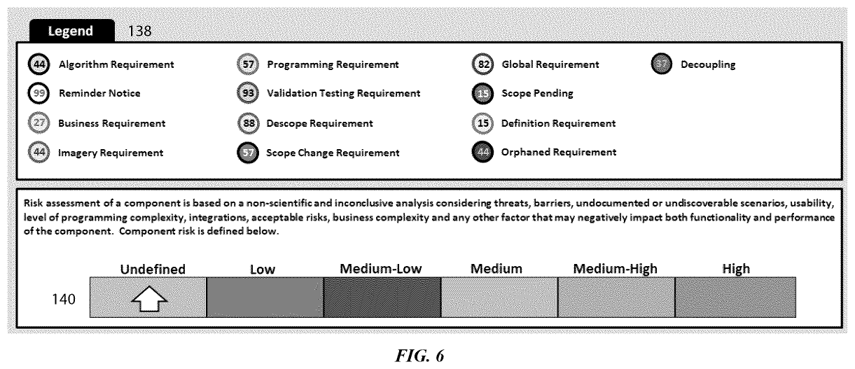

Referring to FIG. 5, at 130 the patient's vitals can be based on the data entry provided by the HCWs wherein each time an individual enters vitals, historical vitals can be shown to the HCW. At 132, the HCW can add Infection Occurrences to a patient's chart and then can decide which occurrence will or will not be displayed in the application. At 134, the HCW can enter patient notes directly into the application and any HCW that has access to the patient's medical chart can access the notes which can allow the HCW the ability to create patients that can be included and made part of the report. At 136, an daily living section can be displayed which allows the HCW for a specific date provide information for such as, for example, ambulation, continence, transfer, dressing, feeding independent, bathing, or the like wherein this information can be saved to the system and then output based on the patient's independence or assistance level.

The present invention also relates to a method for consolidated patient data reporting in healthcare facilities. This method involves receiving patient data from multiple healthcare providers across different facilities via a data input module, storing the received patient data in a unified data repository, processing and amalgamating the stored patient data via an algorithmic processing module using artificial intelligence to search unified data repository for pertinent patient data, and generating and presenting a consolidated report that provides real-time updates and summaries of the patient data. In certain embodiments, the multiple healthcare providers can be at least one healthcare provider wherein the HCW can access the system if the healthcare providers has and pays for the system, or the healthcare provider has implemented the system at their facility, if the healthcare facility does not have the system then the HCW cannot access the system at the facility that does not have the system.

The method can detect and flag any discrepancies or errors in the patient data, transforming processed data to highlight significant trends and vital information in patient care and providing tiered access control with levels including administrative access, facility administration, and patient access. The methodologies used to gather patient data in application system can be design so the system can allow for discrepancies and errors to be identified and corrected into the same web components used to enter data. An incorrect data entry can be identified, and replacement information can be entered easily in any field.

Due to the nature of the system and its ability to view all data and trends while also interpreting data; errors are more thoroughly and easily recognized. If a data point is entered with a human error such as entering “10” instead of the intended entry of “1”, the report can identify this information as a critical value and would populate on the condensed report called Moristat which can then be viewed by a skilled technician as incorrect. The entry can be removed from the report and then entered into the correct component which overrides previously entered information. Additionally, there are component tools that allow healthcare workers to communicate and request changes or clarification to be made to patient records directly through the application interface. The live communication of healthcare workers inside the application has accountability built in which can mean that no question goes unseen, and no task is lost in threads of communication.

The present invention further relates to a report generation system. This system comprises an interface for receiving patient data from healthcare providers and a processing module configured to process the received data and generate a consolidated patient data report in real-time. The report includes summaries and significant trends of the patient data, providing valuable insights into patient care. The system can include a module for accessing historical documents related to the patient data, enhancing the depth and breadth of the data used in the generation of the reports.

The system's technical innovations provide measurable improvements in healthcare data processing through AI-driven analysis of unstructured data within various medical document formats, coupled with proprietary ontology and specialized algorithms focusing on decline and terminal markers. The integration of modal-based human-in-the-loop validation enhances accuracy and addresses conflicting data, while standardization to FHIR ensures data integrity and interoperability. The system's ability to cross-validate algorithmically defined related fields enables identification of critical trends and correlations often missed in standard reports, facilitating proactive identification of decline and terminal changes for timely healthcare decision-making.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications, or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

I claim:

1. A system for consolidated patient data reporting in healthcare facilities, comprising:
a data input module configured to receive patient data from multiple healthcare providers across different facilities;
a unified data repository for storing the patient data;
a processing module for processing and amalgamating the stored patient data;
an artificial intelligence module configured to identify significant trends over time in the stored patient data; and
a reporting module for generating and presenting a consolidated patient data report to a user, wherein the report provides real-time updates and summaries of the patient data.

2. The system of claim 1, wherein the consolidated patient data report contains a tag for each data point that indicates the source of the data point and wherein the tag allows the user to review the precise location of the source of the data point to allow the user to review any anomalies detected in the consolidated patient data report.

3. The system of claim 1, wherein the patient data includes demographics, allergies, weight and height history, infection occurrences, laboratory results, medical imaging results, vital signs, respiratory status, activities of daily living, symptom tracking, diagnosed diseases, hospitalizations, emergency service admissions, communications, historical documents, and general health trends.

4. The system of claim 1, wherein the reporting module transforms processed data to highlight significant trends and vital information in patient care.

5. The system of claim 1, further comprising a security and access control module, wherein the module provides tiered access control with levels including administrative access, facility administration, and patient access.

6. A method for consolidated patient data reporting in healthcare facilities, comprising:
receiving patient data from multiple healthcare providers across different facilities via a data input module;
storing the received patient data in a unified data repository;
processing and amalgamating the stored patient data via an algorithmic processing module using artificial intelligence to identify significant trends over time in the stored patient data;
tagging conflicting and/or redundant data with a tag links to the precise source of the conflicting and/or redundant data to allow clinician review;
generating and presenting a consolidated report that provides real-time updates and summaries of the patient data; and
allowing a clinician to edit reviewed data and presenting the edited information in the consolidated report.

7. The method of claim 6, wherein the patient data is categorized into groups including one or more of demographics, allergies, weight and height history, infection occurrences, laboratory results, medical imaging results, vital signs, respiratory status, activities of daily living, symptom tracking, diagnosed diseases, hospitalizations, emergency service admissions, communications, historical documents, and general health trends, and wherein the method further comprises identifying at least one significant trend in at least one group.

8. The method of claim 7, further comprising identifying relationships between identified significant trends in different groups and using those relationships to generate the consolidated report.

9. The method of claim 6, further comprising providing tiered access control with levels including administrative access, facility administration, and patient access.

10. The method of claim 6 further comprising identifying duplicate data points and consolidating redundant data points in the consolidated report.

11. The method of claim 6 further comprising identifying duplicate data points with a tag that allows the clinician to review and edit the data to consolidate redundant data points.

12. The method of claim 6 further comprising allowing the clinician to identify data points that are clinically conflicting or fabricated, and allowing the clinician to edit or exclude identified data points from the consolidated report.

13. The method of claim 6 further comprising having an AI document parser extract raw data from documents, and analyzing and filtering the extracted data to store the data in the unified data repository.

14. The method of claim 6 further comprising employing conflict resolution workflows to maintain data integrity.

15. A method for providing a patient report, comprising:
receiving patient data from multiple healthcare providers across different facilities;
identifying structured data and unstructured data in the patient data;
extracting raw data from the unstructured data using an AI document parser;
analyzing and filtering the extracted raw data into structured data;
storing the received patient data as structured data in a unified data repository;
processing and amalgamating the stored patient data via an algorithmic processing module using artificial intelligence to search unified data repository for significant trends over time in the patient data;
generating and presenting a consolidated report that provides real-time updates and summaries of the patient data.

16. The method of claim 15, wherein the patient data is categorized into groups including one or more of demographics, allergies, weight and height history, infection occurrences, laboratory results, medical imaging results, vital signs, respiratory status, activities of daily living, symptom tracking, diagnosed diseases, hospitalizations, emergency service admissions, communications, historical documents, and general health trends, and wherein the method further comprises identifying at least one significant trend in at least one group.

17. The method of claim 15, further comprising identifying relationships between identified significant trends in different groups and using those relationships to generate the consolidated report.

18. The method of claim 15, further comprising providing tiered access control with levels including administrative access, facility administration, and patient access.

19. The method of claim 15 further comprising allowing a clinician to edit reviewed data and presenting the edited information in the consolidated report.

20. The method of claim 15 further comprising employing conflict resolution workflows to maintain data integrity.
